(19) United States
(12) Patent Application Publication
(10) Pub. No.: US 2005/0137515 A1
(43) Pub. Date: Jun. 23, 2005

(54) Double Width Offset Length Swab

(76) Inventor: Lonnie Dee King, North Richland Hills, TX (US)

Correspondence Address: LONNIE D. KING, 7232 POSTOAK DR., NORTH RICHLAND HILLS, TX, 77180, US

(21) Appl. No.: 10/707,603

(22) Filed: Dec. 23, 2003

(51) Int. Cl. A61M 35/00

(52) U.S. Cl. 604/1

(57) ABSTRACT

A swab is detailed which is formed of two components: a straight, flat, wide plastic stick with a cutout at each end providing an offset length; and an absorbent material, either cotton or foam rubber, which is attached to each end of the stick completing the swab construction. This design provides a safer and more versatile alternative to traditional swab design.

### BRIEF DESCRIPTION OF DRAWINGS

FIG. 1 shows the completed swab.

FIG. 2 is a cross section view showing the relative position of the absorbent materials at each end of the body frame (stick) to the body frame itself.

FIG. 3 shows an end view of the cut body frame stick evidencing the rounded edges of the body frame material.

FIG. 4 provides a top view of the body frame stick lying flat. It also shows the offset created by the single cutout at lengths along the plastic strip.

FIG. 5 provides a detailed view of the cutting process and the resultant offset created by the single cutout at lengths along the plastic strip.

FIG. 6 shows the shape of the cutting blade used to separate the plastic strip into individual swab body frames (sticks).

### DETAILED DESCRIPTION

The traditional swab is comprised of 2 primary components: a body frame (typically a stick fashioned from either paper, plastic or wood) and an applicator (typically an absorbent material (usually cotton, foam rubber or other synthetic material) attached at one or both ends of the body frame). Generally, the applicator material is attached to the body frame with a small amount of adhesive. The intention of the applicator pads is to absorb fluids, clean wounds and other areas, as well as being used as an applicator for medicines, cosmetics and the like. In this regard, the invention described herein is similar to other traditional swabs.

A well known issue with the traditional swab design relates to the potential danger of damage to the eardrum when swabs are improperly used to clean the outer ear, as referenced in U.S. Pat. No. 5,127,899 (Schmerse, Jr.).

The goal of this instrument is to provide a major improvement over traditional cotton swabs in terms of safety and versatility, and suggest a solution to the above stated issue while maintaining a similar cost of manufacture in relation to more conventional swabs.

Unlike the traditional round single length swab, this device can allow deeper and safer access to areas such the outer ear and opening to the ear canal while greatly reducing or even eliminating the possibility of accidental damage from misapplication of the swab.

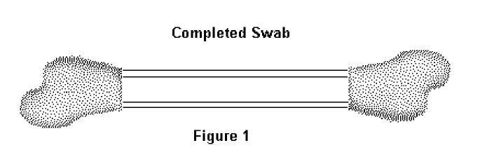

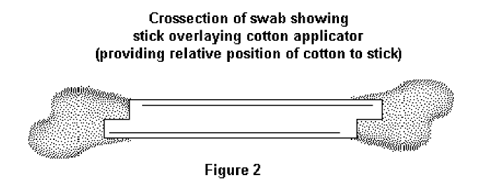

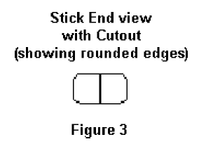

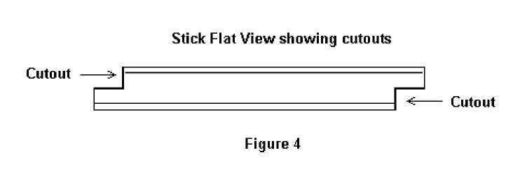

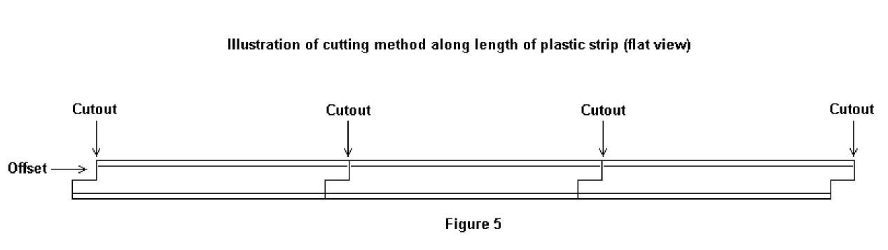

FIGS. 1 through 4 illustrate that this is accomplished by two significant design improvements: 1) the doubling of the body frame width over the round sticks used by traditional swabs, and 2) an offset (distance between rearward and forward portions of the applicator area as shown in FIG. 5) created by a cutout at each end of the body frame. This prevents the extended portion of the swab from intruding beyond a safe distance into the ear canal, providing superior drying or cleaning of this portion of the ear. Additionally, the wider and sturdier design allows for use of the swab in situations where either a standard size swab will not work, or a single swab will not suffice.

A second area of concern regarding traditional designs centers around product and manufacturing costs. While this is a reasonable concern, it is a secondary goal for this design behind providing a safer product than conventional swabs. However, even though this design requires using somewhat more material than a standard swab, the cost of the manufacturing process for this instrument should be comparable to traditional paper based stick designs, which can require rather complex and expensive machinery and manufacturing techniques, as referenced by U.S. Pat. No. 6,080,126 (Zygmont, et al.).

FIG. 5 shows how the stick for this device can be manufactured by inserting a length of flat pre-formed plastic strip with rounded edges into a cutting machine using a shaped cutting blade.

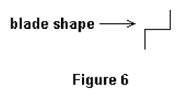

FIG. 6 shows the how the cutting machine stamp-cuts the single cutout along pre-determined and consistent distances along the length of the pre-formed plastic strip creating the offset . This process results in consistent fixed length sticks with practically no waste from the manufacturing process itself. The length of original material could be either in long sticks or from a roll. This material could be either solid or hollow bodied to meet the needs of specific application requirements.

The sticks are then moved down the production line where one of two options can be followed:

1) a small amount of adhesive can be applied to each end of the stick, whereupon the absorbent material is attached to the adhesive covered areas. If using cotton fiber as the applicator material, this would be followed by twisting the stick to wrap the material into the applicator shape. In relation to rolling and applying cotton fiber specifically, please refer to U.S. Pat. No. 3,090,080 (Pellicone et al.), U.S. Pat. No. 3,452,650 (Cobb) and Canadian Patent 990,564 (Cottrell).; or

2) By heating the ends of the cut plastic stick to a temperature high enough for the absorbent material to become attached directly to the semi-melted portions of the stick. This would also lower the materials costs over traditional swab manufacture by eliminating the need for adhesives altogether.

1. A swab comprising two primary components:
a straight, wide, flat elongate body frame (stick) with two ends opposite one another made of pre-formed plastic with rounded edges; and
an applicator, in this case cotton fiber (although other materials such as foam rubber could be used), attached at opposite ends of the body frame.

2. A process for creating the swab including:
Preparing the swab body frame (stick); and
Attaching the applicator material to the body frame.
